(19) United States
(12) Patent Application Publication
(10) Pub. No.: US 2020/0123495 A1
(43) Pub. Date: Apr. 23, 2020

(54) COMPOSITION AND METHOD FOR CONTROLLING INSECTS AND MICROORGANISMS USING PSEUDOMONAS TAIWANENSIS

(71) Applicant: Academia Sinica, Taipei City (TW)

(72) Inventors: Ming-Che Shih, Taipei City (TW); Wen-Jen Chen, Taitung County (TW); Je-Ruei Liu, Taipei City (TW); Yu-Liang Yang, Taipei City (TW)

(21) Appl. No.: 16/664,334

(22) Filed: Oct. 25, 2019

Related U.S. Application Data
(63) Continuation of application No. 15/311,979, filed on Nov. 17, 2016, now abandoned.
(60) Provisional application No. 62/010,776, filed on Jun. 11, 2014.

(51) Int. Cl. C12N 1/20; A01N 63/00; A01N 63/10

(52) U.S. Cl. CPC C12N 1/20; A01N 63/00; C12N 2500/16; C12N 2500/32; C12N 2500/30; A01N 63/10

(57) ABSTRACT

Described herein are methods and compositions for controlling insects and microorganims growth using Pseudomonas taiwanensis and its culture broth.

### CROSS-REFERENCE TO RELATED APPLICATION

This application is a continuation of U.S. application Ser. No. 15/311,979, filed on Nov. 17, 2016, which is the National Stage of International Application No. PCT/US2015/035058, filed on Jun. 10, 2015, which claims priority to U.S. Provisional Application No. 62/010,776, filed on Jun. 11, 2014. The contents of these applications are hereby incorporated by reference herein in their entirety.

### BACKGROUND

Pseudomonas taiwanensis (Pseudomonas sp. TKU015) was classified as a novel bacterium using physiological, biochemical, cellular fatty acid, and 16S rRNA gene sequence method. It was isolated from soils and can grow on medium with shrimp shell powder as the sole carbon and nitrogen source. P. taiwanensis displays high level of extracellular chitinasae, chitosanase, and nattokinase activities under shrimp shell medium. It was shown that recombinant TccC from P. taiwanensis alone could cause mortality of Drosophila larvae, indicating that TccC of P. taiwanensis has its own toxic property.

### SUMMARY

Described herein are methods and compositions for controlling growth of insects and microorganims using Pseudomonas taiwanensis.

In one aspect, described herein is a method of producing a composition for inhibiting growth of a microorganism. The method includes culturing a Pseudomonas taiwanensis strain in a nutrient-limited medium to obtain a culture broth and collecting the culture broth, thus producing the composition. In one embodiment, the medium is an iron-limited medium. The medium can be a M9 minimal medium supplemented with casamino acids, MgSO4, and glycerol. The method can further includes removing cells from the culture broth to obtain a cell-free supernatant and collecting the cell-free supernatant. In one embodiment, the Pseudomonas taiwanensis strain has deposit number DSM 21245. In another embodiment, the Pseudomonas taiwanensis strain has a loss-of-function rpoS mutation. In one embodiment, the microorganism is a phytophathogenic bacterium, a phytophathogenic fungus, or a multidrug resistant bacterium. The microorganism can be Xanthomonas oryzae pv. Oryzae, Colletotrichum gloeosporioides, Phytophthora capsici, Pyricularia oryzae, Rhizoctonia solani, Fusarium oxysporum f sp cattleyae, Staphylococcus epidermidis, Staphylococcus aureus, or Candida albican.

In another aspect, described herein is a composition for inhibiting growth of a microorganism. The composition is produced by the above-described method that includes culturing a Pseudomonas taiwanensis strain in a nutrient-limited medium to obtain a culture broth and collecting the culture broth. The composition can further contain one or more other anti-bacterial, anti-fungal, or insecticidal agents.

In yet another aspect, described herein is a method of inhibiting growth of a microorganism that includes contacting the microorganism with the above-described composition produced by culturing a Pseudomonas taiwanensis strain in a nutrient-limited medium. The microorganism can be a phytophathogenic bacterium, a phytophathogenic fungus, or a multidrug resistant bacterium. In one embodiment, the microorganism is selected from the group consisting of Xanthomonas oryzae pv. Oryzae, Colletotrichum gloeosporioides, Phytophthora capsici, Pyricularia oryzae, Rhizoctonia solani, Fusarium oxysporum f sp cattleyae, Staphylococcus epidermidis, Staphylococcus aureus, or Candida albican.

Also described herein is a method of treating or reducing the risk of rice bacterial blight. The method includes applying the above-described composition to a rice plant in need thereof.

In one aspect, described below is a method of inhibiting growth of a microorganism that includes contacting the microorganism with an isolated pyoverdine having the structure of Q-DSer-Lys-OHHis-aDThr-Ser-cOHOrn. Q is a chromophore and the microorganism is a phytophathogenic bacterium, a phytophathogenic fungus, or a multidrug resistant bacterium. In one embodiment, the microorganism is selected from the group consisting of Xanthomonas oryzae pv. Oryzae, Colletotrichum gloeosporioides, Phytophthora capsici, Pyricularia oryzae, Rhizoctonia solani, Fusarium oxysporum f sp cattleyae, Staphylococcus epidermidis, Staphylococcus aureus, or Candida albican.

In another aspect, described herein is a method of inhibiting growth of an insect that includes contacting the insect with a composition containing a Pseudomonas taiwanensis strain, a Pseudomonas taiwanensis cell lysate, or a Pseudomonas taiwanensis TccC polypeptide. The insect is a Lepidopteran species. In one embodiment, the insect is Plutella xylostella, Spodoptera exigua, or Trichoplusia ni. In one embodiment, the cell lysate is a whole cell lysate or a soluble lysate. The Pseudomonas taiwanensis strain can be cultured in a nutrient-rich medium and the cell lysate can be obtained from a Pseudomonas taiwanensis strain cultured in a nutrient-rich medium.

The details of one or more embodiments are set forth in the accompanying drawings and the description below. Other features, objects, and advantages of the embodiments will be apparent from the description and drawing, and from the claims.

### BRIEF DESCRIPTION OF DRAWINGS

FIG. 1 is a set of a schematic and a graph showing the structure of a pyoverdine and the characteristic ions in the ESI Orbitrap mass spectrum.

FIG. 2 is a schematic comparison of the pyoverdine genes loci of (a) P. taiwanensis and (b) P. aeroginsa POA1

FIG. 3 is a set of (a) graphs showing subcellular localizations of mature pyoverdine and (b) a schematic representation of a pyoverdine secretion pathway in Pseudomonas taiwanensi.

FIG. 4 is a set of (A) graph showing TccC expression levels during different growth phases (grey bars) of P. taiwanensis as compared with that of the internal control 16S rRNA gene (white triangle). Growth curves of P. taiwanensis were measured at OD600 (black circle) and (B) a photograph showing P. xylostella larvae treated with P. taiwanensis.

FIG. 5 is a set of graphs showing toxicity of P. taiwanensis and various cell fractions towards Spodoptera frugiperda Sf9 insect cells. Survival rates of Sf9 cells (A) after infection of P. taiwanensis wild-type and ΔtccC (MOI=1000) and protein fractions (10 μg/ml) derived from (B) cell lysates, (C) soluble lysates and (D) insoluble lysates of P. taiwanensis. Every well in a 96-well plate contained 5000 Sf9 cells. The results were obtained by XTT proliferation assay after P. taiwanensis infection or protein treatment for 72 h.

FIG. 6 is a schematic of a procedure for separating different protein fractions from a P. taiwanensis culture broth.

### DETAILED DESCRIPTION

Described herein is a method of producing a composition for inhibiting growth of a microorganism. The method includes culturing a Pseudomonas taiwanensis strain in a nutrient-limited medium to obtain a culture broth. The culture broth is collected to obtain the composition.

The nutrient-limited medium can be a medium lacking an iron source, e.g., an iron-limited medium. For example, the medium can be a M9 medium, which can be supplemented with other nutrients (e.g., casamino acids, MgSO4, and glycerol). The strain can be cultured in an iron-limited medium at 25 to 37° C. for 1 to 6 days. The medium can contain a certain low amount of iron, as long as the amount is low enough to allow production of a culture broth that is effective against a target microorganism.

The resulting culture broth can be used as is as a composition for inhibiting growth of a microorganism. Optionally, cells can be removed from the culture broth to obtain a cell-free supernatant, which can be then used as the composition.

Also described herein is a method of inhibiting growth of a microorganism using an isolated pyoverdine having the structure of Q-DSer-Lys-OHHis-aDThr-Ser-cOHOrn, wherein Q is a chromophore. Such a pyoverdine can be obtained by culturing a Pseudomonas taiwanensis strain in an iron-limited medium and isolating the pyoverdine thus produced.

Further, this disclosure includes a method of inhibiting growth of an insect. The method includes contacting the insect with a composition containing a Pseudomonas taiwanensis strain, a Pseudomonas taiwanensis cell lysate, or a Pseudomonas taiwanensis TccC polypeptide.

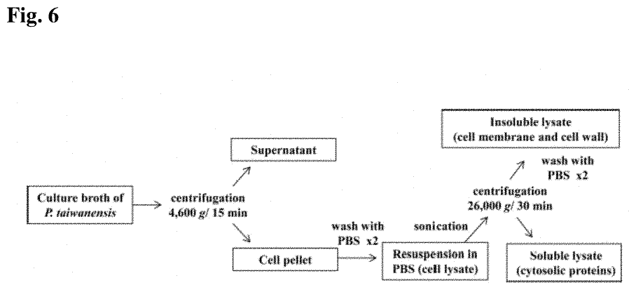

The cell lysate can be a whole cell lysate or a soluble lysate. The cell lysate can be obtained by culturing a Pseudomonas taiwanensis strain in a nutrient-rich medium (e.g., LB medium or ½ TSB medium), disrupting the cells, and then collecting the cell lysate. The cell lysate can be filtered, centrifuged, or otherwise treated to separate the soluable lysate and the insoluble lysate. For example, the procedure shown in FIG. 6 can be used.

A Pseudomonas taiwanensis TccC polypeptide can be obtained using techniques known in the art. Shown below are the nucleic acid sequence (SEQ ID NO:1) and amino acid sequence (SEQ ID NO:2) of a Pseudomonas taiwanensis TccC.

(SEQ ID NO: 1)
TACTCATCTGAGTACGACAGGGATGCCGCCATGCCTGGCGGCTTTTCCGA
TACGTCAAACAGCGCTTTCCGACTAGCAGTCAGCCATACAGCCAAATCAA
GCTGATTCTTCACTCCCCTCTGGGGGTGGCGAAAAATCAACATGATCAAG
GTAACTGCAAGTTGGGACACATAGACTTTTCACTTCATAACGGAACGCCT
ACGGTCACCGTCCGAGACAACCGAGGATTAGGCATCCGCGATATCGCTTA
TCATCGCCATCCCGATACACCCGAACAACTCGACGAACGCATCACCCGCC
ACCGGTTCAACGCCCTTGCGCAGCTTGAGCAAAGCATCGATCCTCGCCTC
CATGAACGCCAAGCCGTGGACGCGACGACCCAACCCAATTACAAATTTCA
TAATTCGCTGACGGGCGATGTCCTGCGTAGCGACAGTGCCGACGCGGGCG
TCACGCTCTCGCTCAACGATGTTCACGGCCGCCCGTGCCTGAGCATTGGC
GCCACAGGCGCGCTCCATCGCTGGCACTATGAAACCCCACCGCTTGCAGG
GCGATTGCTACACGTGAGTGAGCATATCGCCGAAGCAAATCCGCGCATCA
CAGAACGCTTGGTCTGGGGCGACAACACCCAGACTGCGAAGGATCAGAAT
CTTGCAGGCCGATGCGTGCGCCACTATGACACGGCAGGTTGTTGGCAGAT
GGACAGCGCCGGCTTGTCCGCAAGCGTACTTTCCGCCACCCAAAAGTTGC
TGGCGGAAGGCACCGAAGCCGATTGGCAGGGAGAGGACGCGGCAGTCTGG
GACAAGCTACTAGCACCGGATGCGTTCACGACCTCACATCGTATCGACGC
GACGGGAGCTTCCATCGAGCAACGCGATGCGCTCGGCCATACCCAATGCC
AGGCCTATGACATAGCGGGCATGCTGCGTAGCACTCGGCTGATCATGAAA
GGTGGAACGACGCGGGTTATCTTGAAGGCTGTGGAATACTCCGCGTTCGG
ACAAAAGCTGCGGGAAGAACAGGGCAACGGCGTCATTACCACCTACACCT
ACGAACAGCGGACTCAGCGCCTCCTAGGCAGCAAGATCGAACGACGTGCC
GGGCGCAGCGAGGCGAAAGTCCTGCAAGACATACGGTACGAATATGACCC
AGTCGGTAATATCCTGAGTGTGCACAATGATGCGGAGGCGACGCGGTTCT
GGCGTAATCAGAAGATCGTACCGGTCAATCGCTATGCATATGACAGCCTC
TATCAACTGATCTCAGCCAGCGGCCGTGAAATGGCCGATATGCCCCGCCA
AGGCCCTAAGCCTCCCTCCCCCACCATTCCACTCCCGACCAACGACGGGG
CCTACACCAACTACACACGTCGTTACCAATACGATCGCGCTGGCAACCTG
ACGCGTATCTCACACAGCGCACCCGCCTCCAACAACAGCTATACCCTGGA
CATGACAGTGTCCAACCGCAGCAATCGGGCGGTATTGCATACTCTCGCCG
ACGATCCTGCCAAGGTCGATGCCCTCTTCGATGCAGCGGGCAATCAGTTA
CAACTGCAACCTGGCCAATCCCTTCATTGGACACCGCGCGGGCAGCTCGG
CAAGTTCGTGTCACAGGCAGGTGATGACAGCGCTGTTGACCAGGAAAGCT
ATCGCTACGGTGCAGACGGCCAACGGATCGCCAAATACAACTCCCAACAG
GCAGGCGCCCAAACGGGATACGTACTTTATTTGCCAGGGTTGGAGGTGCG
CGCCCGTTTCAGGGACGATGCGATAAAAGAACTGCTTCACGTGATCACCA
TCGGCGAAGCCGGTAATGCTCAAGTGCGATTACTGCACTGGGAAACCGGC
ACACCGCCAAGTGTCAGCAATGACTCGCTGCGCTACGGCTATTCCAATCT
CATAGACAGCGTCGGGCTCGAACTCGATAGCGACGGTCAAATCATCAGCT
ATGAAGAGTACTACCCCTACGGCGGCTCTGCGGTATGGGCCGCTCGCAGC
CAGACAGAAGCCGATTACAAGACCGTGCGTTATTCAGGGAAAGAACGCGA
TGGCACGGGGCTCTATTATTACGGTCACCGGTATTACCAACCCTGGGTCG
GGCGCTGGCTCAGTGCAGACCCTGCCGGTACGGTCGATGGACTCAATCTC
TACCGAATGGTACGAAACAACCCCATTGCCTTAAAAGACAACAACGGATT
GAATGCCGAAGGGTATTACCATGAGTTCCAAGCGCTGAAGAGCGCACCCA
GTATGATCCGTAATACCAGGCTTCAAATTCAAGATTATATGCGAAGCCAA
ACCGAAAGCCGGATTATTTACGTGTTGATGTCGGTCGTTTTGGAAGCGCT
CGCTACGACCATTGGCATGGCCGGCGGCCTCCTGGGCGGTGCGGCGGGAG
GGGCTATAGGAGGCGCTGTAGGAGGGGTTATCGCCAACGTTCCAGGAGCC
GCTGTAGGCGCAACCTGGGGGGCTAGCGTAGGAGGGCTCGTCGGGAAAAC
CGTTGTAAAGAAAGCGGCAGAGAAAATACTCCCGCAGGCTGAGTTGACGC
CAGACCTCGACATGACAAAAAAAATAAACGAAACGGCCGAAGGCGGCCTT
AGGCATAAAATCAAACATTTCCTAGAAAAAGAAATAACCATGGAAAAGCT
CCGTGGAAAAATAACCGATGATCAAATGACCAACGATGCAAACAAAGTGG
CGACAGGCGTGGGTTTACCACAATACCCTCTTACCCTTCCCGTATCAAAA
GCGATAAAAGTCGCCACAGAAGTGGAAAAATCAATAACCGTTACCACAAA
ACATGCAGTAGCCGGGGCAATACCTGCTCAAGTAGAGATTGCAAAAGGTG
CCCTTCATGCCATTTACTCAAAGATAGACGCGCAATTCGGTAAGCTCAGC
AGCATGCGCAGCCGTAAAAGCCTGTTGAGGCCTTTCATACCCGATGGCCC
ACGAGAGCTTTCCATTACATTGAATAATGACCCGTTCAACCCTGATGCAT
GGGTGGGAAGATCGGAGGTCGAGAAGCCTTACCAGGCAGCCTTGGCCGAA
CTGGATAAACTTAACGAACTGTACGTTAAGTACGAAAAAAAATTTCGTAC
TTAAGCGATCTCAACAACCGGCCCCGCCGGTTTGCTGCATGCAAGACCGG
CGGTACCCCAATGCCTGAACTCACCCCGCCTCAGCCCGAATCCGTATCGC
ATCATGACGCCAATATTCCAGGTCACAGTCGATCAGATGCCCATACTGGT
CGCTGTTGACCCGGGTGACATGCAACCCCGGACTACCCGCCGAGACCTTG
AGGGCTGCGGCAGCCGGAGCCGGCAACGCGGTCGGCAGGATCTCGAAGCA
TACCCGGCCGTAAGCGATCCCATAGGCTTTGGCATAGATCTCGGTCAGCG
ACTGACCAAGATCCAACTCCAGGATCCCAGGAAAATACCTAGGGTTCAGG
TAATGCTCGGCATACAGCACCGCGCGCCCGTCGATACGCCGCAAGCGGCA
GATCTGCACCACGCTGGACAAC
(SEQ ID NO: 2)
MGHIDFSLHNGTPTVTVRDNRGLGIRDIAYHRHPDTPEQLDERITRHRFN
ALAQLEQSIDPRLHERQAVDATTQPNYKFHNSLTGDVLRSDSADAGVTLS
LNDVHGRPCLSIGATGALHRWHYETPPLAGRLLHVSEHIAEANPRITERL
VWGDNTQTAKDQNLAGRCVRHYDTAGCWQMDSAGLSASVLSATQKLLAEG
TEADWQGEDAAVWDKLLAPDAFTTSHRIDATGASIEQRDALGHTQCQAYD
IAGMLRSTRLIMKGGTTRVILKAVEYSAFGQKLREEQGNGVITTYTYEQR
TQRLLGSKIERRAGRSEAKVLQDIRYEYDPVGNILSVHNDAEATRFWRNQ
KIVPVNRYAYDSLYQLISASGREMADMPRQGPKPPSPTIPLPTNDGAYTN
YTRRYQYDRAGNLTRISHSAPASNNSYTLDMTVSNRSNRAVLHTLADDPA
KVDALFDAAGNQLQLQPGQSLHWTPRGQLGKFVSQAGDDSAVDQESYRYG
ADGQRIAKYNSQQAGAQTGYVLYLPGLEVRARFRDDAIKELLHVITIGEA
GNAQVRLLHWETGTPPSVSNDSLRYGYSNLIDSVGLELDSDGQIISYEEY
YPYGGSAVWAARSQTEADYKTVRYSGKERDGTGLYYYGHRYYQPWVGRWL
SADPAGTVDGLNLYRMVRNNPIALKDNNGLNAEGYYHEFQALKSAPSMIR
NTRLQIQDYMRSQTESRITYVLMSVVLEALATTIGMAGGLLGGAAGGAIG
GAVGGVIANVPGAAVGATWGASVGGLVGKTVVKKAAEKILPQAELTPDLD
MTKKINETAEGGLRHKIKHFLEKEITMEKLRGKITDDQMTNDANKVATGV
GLPQYPLTLPVSKAIKVATEVEKSITVTTKHAVAGAIPAQVEIAKGALHA
IYSKIDAQFGKLSSMRSRKSLLRPFIPDGPRELSITLNNDPFNPDAWVGR
SEVEKPYQAALAELDKLNELYVKYEKKFRT

One or more additional insectidal, anti-fungal, or anti-bacterial agents can be added to the compositions produced by the methods described herein or used in the methods described herein. Such agents include, but are not limited to, streptocycline (streptomycin sulphate and tetracycline, e.g., 10%), Tecloftalam (e.g., 10%), Probenazole (e.g., 6% or 10%), Cartap hydrochloride, aromatic hydrocarbon, guanidine, dicarboximide, 2-aminopyrimidine, organophosorus, benzimidazole, carboxamide, sterol biosynthesis inhibiting, anti-Oomycetes, strobilurin, anilinopyrmidine, phenylpyrrole benzamide, quinolone, and Bt insecticidal toxins.

Other agents, such as inactive ingredients (e.g., preservatives, carriers, solvents, and dyes), can also be included in the composition.

The Pseudomonas taiwanensis strain used in the methods described herein can be the strain having deposit number DSM 21245. The strain can also be a mutant strain having a loss-of-function rpoS mutation. Such a strain can be generated using recombinant and/or genetic techniques known in the art. The nucleic acid sequence (SEQ ID NO:3) and amino acid sequence (SEQ ID NO:4) of a Pseudomonas taiwanensis rpoS are shown below:

(SEQ ID NO: 3)
ATGGCTCTCAGCAAAGAAGTGCCGGAGTTTGACATCGACGATGACCTCCT
GTTGATGGAGACGGGCATCGTTTTGGAAACGGATGTGGTGTCAGACGAAC
CTGCTGTACCTTCGGTTCGGACCAAGGCCAAACAAGGCTCATCGCTCAAA
CAGCACAAGTACATCGATTACAGCCGGGCGCTCGACGCCACCCAGCTGTA
TCTCAACGAAATCGGCTTTTCTCCGCTGCTCTCCCCCGAAGAGGAAGTGC
ATTACGCACGCCTGTCGCAAAAAGGCGATCCGGCTGGCCGTAAGCGCATG
ATCGAGAGCAACCTGCGCCTGGTGGTCAAGATTGCGCGCCGCTACGTCAA
TCGTGGCCTGTCGCTACTCGACCTGATCGAAGAGGGCAACCTCGGTCTGA
TCCGCGCGGTAGAAAAGTTCGATCCGGAGCGCGGTTTCCGTTTCTCGACC
TATGCGACCTGGTGGATTCGCCAGACCATCGAACGGGCGATCATGAACCA
GACCCGCACCATCCGCCTGCCGATCCACGTGGTCAAGGAGCTCAACGTCT
ACCTGCGTGCCGCGCGGGAGCTGACCCAGAAGCTCGACCACGAGCCTTCC
CCGGAAGAAATCGCCGGGCTTTTGGAAAAACCCGTGGCCGAGGTCAAGCG
CATGCTTGGGCTCAACGAGCGTGTCTCTTCGGTGGACGTTTCTCTCGGCC
CGGACTCCGACAAGACCCTGCTCGACACGCTGACGGACGATCGCCCGACC
GACCCGTGCGAGCTGCTGCAGGACGACGACCTCTCCCAGAGCATCGACCA
ATGGCTGGGTGAGTTGACCGACAAGCAGCGTGAGGTGGTGGTGCGTCGGT
TCGGCTTGCGGGGCCACGAAAGCAGCACCCTTGAGGATGTAGGCCTGGAA
ATCGGCCTGACCCGAGAGCGCGTGCGGCAGATCCAGGTCGAGGGGCTCAA
GCGTCTACGTGAAATCCTTGAAAAGAACGGCCTCTCGAGTGAGTCGCTGT
TCCAGTAA
(SEQ ID NO: 4)
MALSKEVPEFDIDDDLLLMETGIVLETDVVSDEPAVPSVRTKAKQGSSLK
QHKYIDYSRALDATQLYLNEIGFSPLLSPEEEVHYARLSQKGDPAGRKRM
IESNLRLVVKIARRYVNRGLSLLDLIEEGNLGLIRAVEKFDPERGFRFST
YATWWIRQTIERAIMNQTRTIRLPIHVVKELNVYLRAARELTQKLDHEPS
PEEIAGLLEKPVAEVKRMLGLNERVSSVDVSLGPDSDKTLLDTLTDDRPT
DPCELLQDDDLSQSIDQWLGELTDKQREVVVRRFGLRGHESSTLEDVGLE
IGLTRERVRQIQVEGLKRLREILEKNGLSSESLFQ

Any of the compositions and methods described above can be used to inhibit the growth of various insects and microorganisms (e.g., phytophathogenic bacteria, phytophathogenic fungi, or multidrug resistant bacteria. They can also be used to treat or reduce the risk of diseases caused by the insects and microorganisms, e.g., rice bacterial blight caused by Xanthomonas oryzae pv. Oryzae. For example, the compositions can be administered to (e.g., sprayed on) infected or uninfected targets (e.g., rice plants).

The microorganisms include, but are not limited to Xanthomonas oryzae pv. oryzae (Xoo), Xanthomonas oryzae pv. oryzicola (Xoc), Colletotrichum acutatum, Colletotrichum agaves, Colletotrichum alcornii, Colletotrichum arachidis, Colletotrichum baltimorense, Colletotrichum capsici, Colletotrichum caudatum, Colletotrichum cereal, Colletotrichum coccodes, Colletotrichum crassipes, Colletotrichum dematium, Colletotrichum derridis, Colletotrichum destructivum, Colletotrichum fragariae, Colletotrichum gloeosporioides, Colletotrichum gossypii, Colletotrichum graminicola, Colletotrichum higginsianum, Colletotrichum kahawae, Colletotrichum lindemuthianum, Colletotrichum lini, Colletotrichum mangenotii, Colletotrichum musae, Colletotrichum nigrum, Colletotrichum orbiculare, Colletotrichum pisi, Colletotrichum somersetense, Colletotrichum sublineolum, Colletotrichum trichellum, Colletotrichum trifolii, Colletotrichum truncatum, Colletotrichum viniferum, Colletotrichum zoysiae, Phytophthora taxon Agathis, Phytophthora alni, Phytophthora boehmeriae, Phytophthora botryose, Phytophthora brassicae, Phytophthora cactorum, Phytophthora cajani, Phytophthora cambivora, Phytophthora capsici, Phytophthora cinnamomi, Phytophthora citricola, Phytophthora citrophthora, Phytophthora clandestine, Phytophthora colocasiae, Phytophthora cryptogea, Phytophthora drechsleri, Phytophthora diwan ackerman, Phytophthora erythroseptica, Phytophthora fragariae, Phytophthora fragariae var. rubi, Phytophthora Gemini, Phytophthora glovera, Phytophthora gonapodyides, Phytophthora heveae, Phytophthora hibernalis, Phytophthora humicola, Phytophthora hydropathical, Phytophthora irrigate, Phytophthora idaei, Phytophthora ilicis, Phytophthora infestans, Phytophthora inflate, Phytophthora ipomoeae, Phytophthora iranica, Phytophthora katsurae, Phytophthora lateralis, Phytophthora medicaginis, Phytophthora megakarya, Phytophthora megasperma, Phytophthora melonis, Phytophthora mirabilis, Phytophthora multivesiculata, Phytophthora nemorosa, Phytophthora nicotianae, Phytophthora PaniaKara, Phytophthora palmivora, Phytophthora phaseoli, Phytophthora pini, Phytophthora porri, Phytophthora plurivora, Phytophthora primulae, Phytophthora pseudosyringae, Phytophthora pseudotsugae, Phytophthora quercina, Phytophthora ramorum, Phytophthora sinensis, Phytophthora sojae, Phytophthora syringae, Phytophthora tentaculata, Phytophthora trifolii, Phytophthora vignae, Pyricularia angulate, Pyricularia apiculata, Pyricularia borealis, Pyricularia buloloensis, Pyricularia caffra, Pyricularia cannae, Pyricularia cannicola, Pyricularia caricis, Pyricularia commelinicola, Pyricularia costi, Pyricularia costina, Pyricularia curcumae, Pyricularia cyperi, Pyricularia didyma, Pyricularia digitariae, Pyricularia distorta, Pyricularia dubiosa, Pyricularia ebbelsii, Pyricularia echinochloae, Pyricularia euphorbiae, Pyricularia fusispora, Pyricularia globbae, Pyricularia grisea, Pyricularia guarumaicola, Pyricularia juncicola, Pyricularia kookicola, Pyricularia lauri, Pyricularia leersiae, Pyricularia longispora, Pyricularia lourinae, Pyricularia luzulae, Pyricularia occidentalis, Pyricularia oncosperma, Pyricularia oryzae, Pyricularia panici-paludosi, Pyricularia parasitica, Pyricularia penniseti, Pyricularia peruamazonica, Pyricularia pyricularioides, Pyricularia rabaulensis, Pyricularia sansevieriae, Pyricularia scripta, Pyricularia setariae, Pyricularia sphaerulata, Pyricularia submerse, Pyricularia subsigmoidea, Pyricularia vandalurensis, Pyricularia variabilis, Pyricularia whetzelii, Pyricularia zingiberis, Pyricularia zizaniicola, Rhizoctonia bataticola, Rhizoctonia carotae, Rhizoctonia cerealis, Rhizoctonia crocorum, Rhizoctonia fragariae, Rhizoctonia goodyerae-repentis, Rhizoctonia leguminicola, Rhizoctonia oryzae, Ceratorhiza ramicola, Rhizoctonia zeae, Fusarium oxysporum f.sp. albedinis, Fusarium oxysporum f.sp. asparagi, Fusarium oxysporum f.sp. batatas, Fusarium oxysporum f.sp. betae, Fusarium oxysporum f.sp. cannabis, Fusarium oxysporum f.sp. cepae, Fusarium oxysporum f.sp. ciceris, Fusarium oxysporum f.sp. citri, Fusarium oxysporum f.sp. coffea, Fusarium oxysporum f.sp. cubense, Fusarium oxysporum f.sp. cyclaminis, Fusarium oxysporum f.sp. herbemontis, Fusarium oxysporum f.sp. dianthi, Fusarium oxysporum f.sp. lactucae, Fusarium oxysporum f.sp. lentis, Fusarium oxysporum f.sp. lini, Fusarium oxysporum f.sp. lycopersici, Fusarium oxysporum f.sp. medicaginis, Fusarium oxysporum f.sp. melonis, Fusarium oxysporum f.sp. nicotianae, Fusarium oxysporum f.sp. niveum, Fusarium oxysporum f.sp. palmarum, Fusarium oxysporum f.sp. passiflorae, Fusarium oxysporum f.sp. phaseoli, Fusarium oxysporum f.sp. pisi, Fusarium oxysporum f.sp. radicis-lycopersici, Fusarium oxysporum f.sp. ricini, Fusarium oxysporum f.sp. strigae, Fusarium oxysporum f.sp. tuberosi, Fusarium oxysporum f.sp. tulipae, Fusarium oxysporum f.sp. vasinfectum, Staphylococcus arlettae, Staphylococcus agnetis, Staphylococcus aureus, Staphylococcus auricularis, Staphylococcus capitis, Staphylococcus caprae, Staphylococcus carnosus, Staphylococcus caseolyticus, Staphylococcus chromogens Staphylococcus cohnii, Staphylococcus condiment, Staphylococcus delphini, Staphylococcus devriesei, Staphylococcus epidermidis, Staphylococcus equorum Staphylococcus felis, Staphylococcus fleurettii, Staphylococcus gallinarum, Staphylococcus haemolyticus, Staphylococcus hominis, Staphylococcus hyicus, Staphylococcus intermedius, Staphylococcus kloosii, Staphylococcus leei, Staphylococcus lentus, Staphylococcus lugdunensis, Staphylococcus lutrae, Staphylococcus massiliensis, Staphylococcus microti, Staphylococcus muscae Staphylococcus nepalensis, Staphylococcus pasteuri, Staphylococcus pettenkoferi Staphylococcus piscifermentans, Staphylococcus pseudintermedius, Staphylococcus pseudolugdunensis, Staphylococcus pulvereri, Staphylococcus rostri, Staphylococcus saccharolyticus, Staphylococcus saprophyticus, Staphylococcus schleiferi Staphylococcus sciuri, Staphylococcus simiae, Staphylococcus simulans, Staphylococcus stepanovicii, Staphylococcus succinus, Staphylococcus vitulinus, Staphylococcus warneri, Staphylococcus xylosus, Candida albicans, Candida ascalaphidarum, Candida amphixiae, Candida Antarctica Candida argentea, Candida atlantica, Candida atmosphaerica, Candida blattae Candida bromeliacearum, Candida carpophila, Candida carvajalis, Candida cerambycidarum, Candida chauliodes, Candida corydalis, Candida dosseyi, Candida dubliniensis, Candida ergatensis, Candida fructus, Candida glabrata, Candida fermentati, Candida guilliermondii, Candida haemulonii, Candida insectamens, Candida insectorum, Candida intermedia, Candida jeffresii, Candida kefyr, Candida keroseneae, Candida krusei, Candida lusitaniae, Candida lyxosophila, Candida maltose, Candida marina, Candida membranifaciens, Candida milleri, Candida oleophila, Candida oregonensis, Candida parapsilosis, Candida quercitrusa, Candida rugosa, Candida sake, Candida shehatea, Candida temnochilae, Candida tenuis, Candida theae, Candida tolerans, Candida tropicalis, Candida tsuchiyae, Candida sinolaborantium, Candida sojae, Candida subhashii, Candida viswanathii, Candida utilis, and Candida ubatubensis.

The insects include those of the Lepidopteran species, e.g., Plutella xylostella, Spodoptera exigua, and Trichoplusia ni.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications cited herein are hereby incorporated herein by reference in their entirety.

### Example 1: Type VI Secretion System-Mediated Secretion of Pyoverdine from Pseudomonas taiwanesis Inhibits Growth of Rice Pathogen Xanthomonas oryzae pv. Oryzae

Rice bacterial blight caused by Xanthomonas oryzae pv. oryzae (Xoo) is one of the most destructive diseases of rice throughout the world. We showed that P. taiwanensis displayed strong antagonistic activity against Xoo. Using MALDI-TOF imaging mass spectrometry (MALDI-IMS), we identified a pyoverdine secreted by P. taiwanensis that can inhibit the growth of Xoo. Through Tn5 mutagenesis of P. taiwanensis, we showed that mutations in genes that encode components of Type VI secretion system (T6SS) and pyoverdine biosynthesis and maturation resulted in reduced toxicity against Xoo. Our data demonstrated that pyoverdine can be secreted into culture medium via T6SS to inhibit growth of Xoo. Our data thus differ from studies reporting that delivery of effectors by T6SS requires physical contact between donors and recipients.

### Anti-Xoo Activity and Identification of Related Genes by Genome-Wide Mutagenesis

We tested several Pseudomonas species to search for potential biocontrol agents against Xoo. P. taiwanensis displayed highest anti-Xoo activity when it was grown on iron-limited medium as compared to nutrient rich media (LB and ½ TSB). Among these media, P. taiwanensis had similar growth rates. In contrast to P. taiwanensis, P. syringae DC3000 did not exhibit toxicity against Xoo.

To identify factors that affect the antagonistic activity of P. taiwanensis against Xoo, we generated a Tn5 mutagenized library of P. taiwanensis and screened for mutants with attenuated antagonistic activities against Xoo. The insertion sites of mutants were determined using TAIL-PCR. Among these mutants, we found 4 mutants whose growth were not affected and displayed attenuated antagonistic activity against Xoo. These mutants had insertion sites in genes that encode T6SS (clpV), pyoverdine synthetase (pvdL), pyoverdine translocation and maturation (pvdE), and regulator (rpoS).

ATPase ClpV is an important component of the T6SS apparatus and contributes to VipA/VipB tubules remodeling. See Bonemann et al., EMBO J 28, 315-325 (2009). PvdL is a peptide synthetase involved in the biosynthesis of pyoverdine chromophore. See Mossialos et al., Mol Microbiol 45, 1673-1685 (2002). PvdE is a cell membrane protein involved in translocation of pyoverdine precursors to periplasma. See Ravel and Cornelis, Trends Microbiol 11, 195-200 (2003). No significant difference in growth between wild type (WT) and mutant strains (ΔclpV and ΔpvdL) was detected from 4 h (lag phase) to 72 h (death phase) in iron-limited LP broth.

In antagonistic assays, whole culture or cell-free culture supernatants of wild-type P. taiwanensis showed substantial toxicity against Xoo. In contrast, whole culture or cell-free supernatants of ΔclpV showed lower toxicity as compared to WT. Both ΔpvdL and ΔpvdE mutants exhibited no toxicity toward Xoo.

Characterization of P. taiwanensis Pyoverdine Toxicity Against Xoo and its Secretion by T6SS

We used MALDI-IMS to survey the secreted metabolites from wild-type and mutants of P. taiwanensis on the surface of agar plates to survey the secreted metabolites and compounds from P. taiwanensis on the surface of agar plate. A signal with m/z 1044 was detected in plates with wild-type P. taiwanensis, whereas the level of m/z 1044 in ΔclpV was much lower than that of wild-type. However, no m/z 1044 compound was detected around ΔpvdL and ΔpvdE, which suggest m/z 1044 is a pyoverdine analogue.

The pyoverdine were purified using a Cu-sepharose column and checked by MADLI-IMS. The fluorescent pyoverdine with the strongest absorbance at 400 nm was monitored by an UV detector in HPLC analysis. The supernatants from cultures of ΔclpV mutant had a lower concentration of pyoverdine than wild-type. Quantification using LC-MS showed that the pyoverdine level in wild-type is about 2-filed higher than in the ΔclpV mutant. We did not detect pyoverdine in the culture supernatants of the ΔpvdL and ΔpvdE mutants.

Several studies have characterized T6SS-mediated antibacterial activities in Pseudomonas aeruginosa, Vibrio cholera and Burkholderia thailandensis. These studies showed that the antibacterial effector proteins were injected through T6SS directly into target cells through cell-cell contact. In our study, the culture supernatant of wild-type P. taiwanensis displayed higher toxicity against Xoo than that of the T6SS mutant ΔclpV, suggesting that T6SS-mediated secretion of anti-Xoo compounds does not require cell-cell contact.

To verify that the clp Vmutation affected T6SS activity in P. taiwanensis, two experiments were performed. First, western blot analysis was used to quantify the level of VgrG protein, which is a biomarker for T6SS activity, in cell-free culture supernatant. The results showed that VgrG could be detected in cell-free culture supernatants of wild-type and clpV complemented stain ΔclpV/clpV. In contrast, no significant level of VgrG could be detected in the culture supernatant of the clpV mutant. The results also showed that the levels of VgrG in the cell lysates are similar between wild-type, ΔclpV, and ΔclpV/clpV. RNA polymerase α-subunit RpoA was used as a loading control. These results demonstrate that the clp Vmutant is defective in T6SS function and introduction of a wild-type clp Vgene into this mutant could restore T6SS function. These results indicate that in P. taiwanensis T6SS is involved in anti-Xoo activity by secreting pyoverdine into the medium. Second, we performed a complementation test by introducing a wild-type copy of the clp Vgene into the Δclp Vmutant.

In the MALDI-IMS assays, the introduction of wild-type clpV restored the secreted level of pyoverdine in the culture supernatant. The data indicated that the reduced secretion of pyoverdine in the ΔclpV mutant resulted from a mutation in the clpV locus.

To demonstrate the anti-Xoo activity of pyoverdine from P. taiwanensis, different concentration of the purified pyoverdine were tested by CAS agar plates assay. CAS reaction rate, which measures the removal of iron by pyoverdine from the CAS dye, was rapidly detected at 1.2 and 1.5 mg pyoverdine reaction on CAS agar plates. After demonstrating pyoverdine activity, inhibition of cell growth (IC₅₀) and lethal dose (LD₅₀) against Xoo were tested. The IC₅₀ of pyoverdine toward Xoo was about 2.035 mg/ml (R²=0.9946). The LD₅₀ was about 1.98 mg/ml (R²=0.9775). The IC₅₀ and LD₅₀ data showed that pyoverdine has anti-Xoo activity.

To further clarify the role of pyoverdine in the antagonistic activity of P. taiwanensis against Xoo, iron-enriched culture media were used to examine pyoverdine activity. The culture broth of P. taiwanensis showed a dose-dependent decrease in toxicity when extra iron was applied to Xoo-containing plates. At higher concentrations of iron (300, 600, and 1000 μM FeCl₃), P. taiwanensis had almost no antagonistic activity toward Xoo. The growth of P. taiwanensis was unaffected by the addition of iron compared to the control (½ TSB only). Together, the results suggest that the antagonistic activity of pyoverdine against Xoo is via an iron-competition mechanism. We propose that when there is a limited amount of iron in the environment, P. taiwanensis competes efficiently for iron by secreting pyoverdine to chelate iron and take up pyvoverdine-iron complexes through PvdRT-OpmQ, which results in retarded growth of Xoo. At higher concentrations of iron, however, the pyvoverdine secreted by P. taiwanensis is not sufficient to absorb all the available iron, which compromises its anti-Xoo activity.

Identification of the Structure, Gene Loci and Function of Pyoverdine in P. taiwanensis

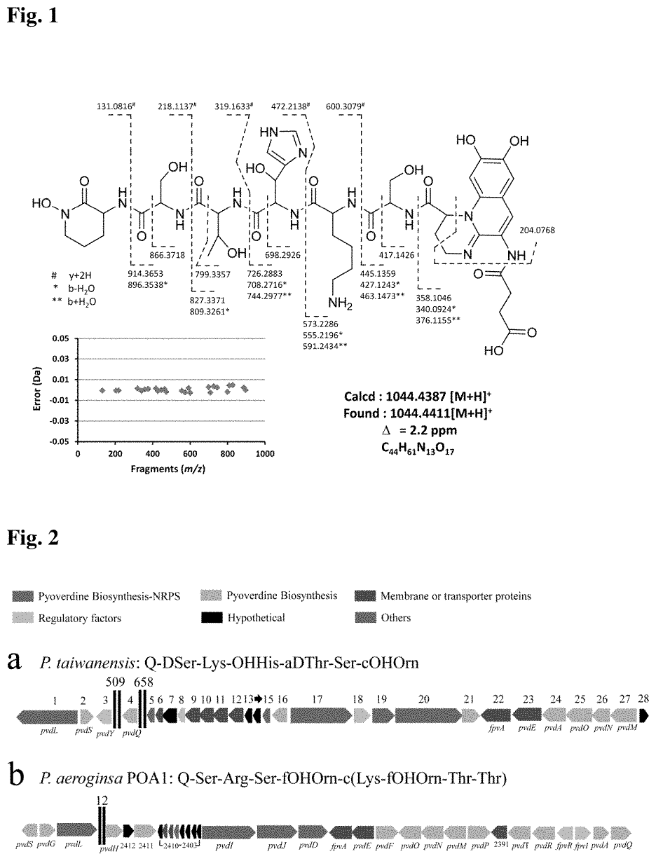

The purified pyoverdine (m/z 1044) was subjected to tandem mass spectrometry to identify primary structure and order of amino acids. See FIG. 1. The order of the amino acid sequence corresponded to a predictor of NRPS adenylation domain specificity (Ser-Lys and Thr-Ser-OH-Orn). This pyoverdine from P. taiwanensis is identical to that from P. fluorescens 9AW and P. putida 9BW. See Budzikiewicz et al., Z. Naturforsch. Sect. C 52, 721 (1997).

Pyoverdine contains a variable peptide side chain with different compositions of amino acids, and a conserved fluorescent chromophore. The peptide of pyoverdine side chain is highly variable among fluorescent Pseudomonas species. The biosynthesis and transport of the pyoverdines have been studied extensively in Pseudomonas aeruginosa PAO1. The majority of pyoverdine biosynthetic and transport genes form a cluster in both P. taiwanensis and P. aeruginosa PAO1, whereas the pvdL gene is located in a separate cluster in both species. See FIG. 2. The pvdL gene is involved in synthesis of the conserved fluorescent chromophore of pyoverdine precursor in all Pseudomonad. Homologues of pvdL, pvdJ, and pvdD are involved in biosynthesis of the peptide backbone of pyoverdine. Pyoverdine precursor is transferred into periplasmic space from cytoplasm by PvdE, which is an inner membrane transporter, and then processed into mature pyovedine by PvdA, Q, N, M, O, and P. PvdA is a membrane-bound L-ornithine (Om) N⁸-oxygenase that catalyzes Om hydroxylation. After maturation, the fluorescent pyoverdine is secreted into the extracellular environment. PvdM, pvdN, pvdO, pvdA, and pvdE genes are clustered together in P. taiwanensis.

The syrP gene, which encodes a pyoverdine biosynthesis regulatory protein, is present downstream of pvdl in P. taiwanensis. In contrast, the syrP gene is located in the middle of pvd gene clusters in P. syringae DC3000, P. putida KT2440 and P. fluorescens Pf0-1. SyrP protein functions in the hydroxylation of Asp and is involved in stringomycin E production, which is synthesized by NRPS. However, homologous of syrP was not identified in P. aeruginosa PAO1.

### Characterization of the Role of T6SS in Pyoverdine Secretion

To characterize the role of T6SS in pyoverdine secretion, we used IMS to quantify the pyoverdine secreted in cultures of the clp V mutant and the wild-type. Under iron-limited conditions, pyoverdine (m/z 1044.44) was found around P. taiwanensis colonies after 12 h incubation in time course experiments. At 16 h, the amount of pyoverdine in clpV mutant was much lower than that of wild type on the surface of agar plates. However, pyoverdine was also detected on the agar plates in clpV mutant. This is due to pyoverdine accumulated in the medium after long time incubation, even in wild type and clpV mutant. Cross section IMS of the agar plates showed that the amount of pyoverdine secreted by the clp V mutant after a 36-h incubation was lower than that of the wild-type. On the other hand, IMS data showed that pyoverdine was not stimulated by Xoo.

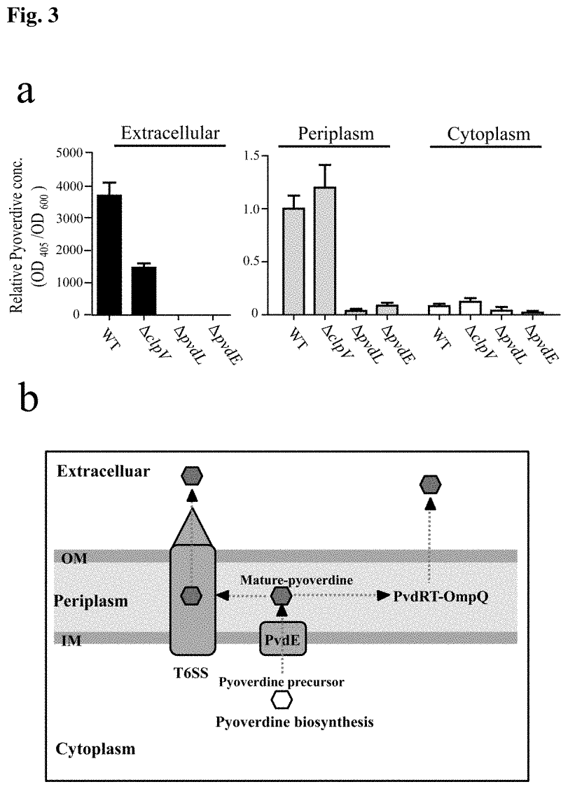

To further evaluate the involvement of T6SS in the secretion of pyoverdines, we quantified mature pyoverdine (fluorescent pyoverdine) in the extracellular supernatants, periplasm, and cytoplasm of the wild-type and the three mutants with defective anti-Xoo activity. See FIG. 3a. In both the wild-type and ΔclpV, the amounts of pyoverdine were highest in the extracellular supernatants, much lower in the periplasm and non-detectable in the cytoplasm. See FIG. 3a. When compared in detail, less pyoverdine was found in the extracellular supernatant of the ΔclpV mutant than the wild-type (left panel, FIG. 3a). In contrast, the ΔclpV mutant accumulated slightly more mature pyoverdine in the periplasm than the wild-type (middle panel, FIG. 3a). No significant quantity of pyoverdine was detected in any of the subcellular fractions of ΔpvdL and ΔpvdE. The data also confirmed that PvdL and PvdE were involved in the biosynthesis and maturation of pyoverdine. Taken together, these results suggest that ΔclpV mutation does not affect intracellular pyoverdine production, but does affect the translocation of pyoverdine from the periplasm to the culture medium.

A schematic of pyoverdine transportation in P. taiwanensis is shown in FIG. 3b. Negative control of pyoverdine expression by RpoS The stationary phase sigma factor, RpoS, is a global stress response regulator. We identified an rpoS P. taiwanensis mutant that exhibited increased pyoverdine production in iron-limited medium. Incubation of the rpoS mutant strain exhibits deep green color under iron-limited medium compared to light green color in wild type after 3 days of flask incubation, and rpoS mutant did not affect cell growth. This is probably because the amount of florescent pigment pyoverdine accumulates in the medium to exhibit deep green color. In antagonistic assay, the rpoS mutant showed a larger inhibition zone toward Xoo than wild type. IMS data showed that the rpoS mutant secreted more pyoverdine than the wild type. Quantification of the pyoverdine showed that the rpoS mutant produced 2-3 fold higher concentration of pyoverdine in iron limited supernatant as compared to the wild type. These results suggested that pyoverdine production is negatively regulated by RpoS in P. taiwanensis.

### Materials and Methods

### (1) Microorganisms and Antagonistic Assay

P. taiwanensis sp. nov. CMS^{T} (=BCRC17751T=DSM 21245T), was isolated from soil and characterized using phenotypic and molecular taxonomic method. See Wang, L. T. et al., International Journal of Systematic and Evolutionary Microbiology 60, 2094-2098 (2009). Xanthomonas oryzae pv. oryzae (Xoo) XF89b strain was isolated from rice blight disease in Taichung of Taiwan. Pseudomonas syringae pv tomato (Pst DC3000) was provided by Laurent Zimmerli from the Institute of Plant Biology, National Taiwan University.

Antagonistic activity of Pseudomonas taiwanensis against rice blight disease Xanthomonas oryzae pv. oryzae (Xoo) was tested on ½ trypticase soya agar (TSB) agar plates (BD Biosciences) at 28° C. P. taiwanensis preculture was grown in an iron-limited medium (M9 minimal medium supplemented with 1% Casamino Acids, 1 mM MgSO4, and 0.5% glycerol) and incubated into 500 ml flask containing 100 ml medium at 28° C. and 200 rpm for 24 h. Xoo preculture was grown in ½ TSB medium at 28° C. for 3 days. Xoo was mixed with melted ½ agar medium before pouring into empty plate. For bioassay, P. taiwanensis (10⁹CFU/ml) or filtered (0.22 μm) supernatant was injected into the hole of Xoo-mixed LB agar plate until the inhibition zones had been characterized.

### (2) Comparison of Pyoverdine (m/z 1044) Levels by LC/MS

After 1 day of incubation, the culture supematants were collected by centrifugation for 10 min at 4500 g. The culture supematants were sterilized through 0.22-μm filter. A 10 mL aliquot of each filtered supernatant was dried by freeze drying and resuspended in 50% methanol. The total number of metabolites was detected by high-resolution liquid chromatography-mass spectrometry (LC/MS) (ESI-Orbitrap, conducted by the Metabolomics Core Facility, Academia Sinica, Taiwan). The peak height and area were determined for calculation of the pyoverdeine level in LC/MS analyses.

### (3) Construction of the Transposon Library

An EZ-Tn5 transposon mutagenesis kit (KAN-2; Epicentre) was used to make a random mutant library. EZ-Tn5 transposon mutagenesis was performed according to the manufacturer's instructions. P. taiwanensis competent cells were prepared according to the method outlined in Choi et al. (J Microbiol Methods 64:391-397, 2006). To screen the Tn5 mutant library, we utilized the P. taiwanensis mutagenesis library to incubate with Xoo, providing the opportunity to find virulence-related genes. The flanking sequences of insertion sites were amplified by TAIL-PCR. Two sets of random primers and the specific regions of the two ends of the transposon primers were designed by Sun et al. (FEMS Microbiol Lett, 226:145-150, 2003). The Tn5 mutant strains of this study were further determined by PCR and sequencing. The mutant strains (clpV, pvdL, pvdE) were determined by UV light. The nucleotide sequence of P. taiwanensis clpV, pvdL, and pvdE were submitted to the GenBank database under accession numbers KM061430, KM036007 and KM036029, respectively. Finally, we used Southern blot analysis to check Tn5-inserted mutants insertion numbers. NcoI- and EagI-digested genomic DNA of Tn5-inserted mutants were analyzed by Southern blot hybridization with a DIG-labelled PCR probe. Southern analysis with a probe of the kanamycin resistance gene was used to confirm insertion number. After hybridization, the Southern blots were developed using a detection kit (Roche).

In order to monitor downstream gene expression of clpV, we detected PT3445 and yhfE gene expression in the WT and clpV mutant by RT-PCR. The result showed that clpV mutation does not affect downstream gene expression. The clpV mutant was complemented by broad host range vector pCPP30 expression. Induction of pCPP30 harboring clpV fragment was performed overnight by adding final 1 mM isopropyl-β-D-thiogalactopyranoside (IPTG) to iron-limited medium.

### (4) Secretory T6SS Component

VgrG was detected in culture supernatant by western blotting to ensure T6SS activity using anti-Agrobacterium tumefaciens VgrG antibody. The RNA polymerase a-subunit RpoA, which was used as a loading control in western blots, was detected using anti-Agrobacterium tumefaciens RpoA antibody. Both anti-VgrG and anti-RpoA antibodies were provided by Dr. Erh-Min Lai, Institute of Plant and Microbial Biology, Academia Sinica, Taiwan. Twenty-four hour culture of P. taiwanensis wild type and clpV mutant in iron-limited medium were grown to an optical density at 600 nm (00600) of ˜0.8. After centrifugation, at 4500 g for 10 min, the culture supernatant was sterilized through 0.22-μm Durapore polyvinylidene fluoride (PVDF) (lowest protein binding) syringe filters. Cell-free culture supernatant proteins (20 ml) were precipitated by adding trichloroacetic acid (TCA) to final 10% TCA concentration overnight at 4° C. and the pellet was washed twice with ice-cold acetone to remove residual TCA. TCA-precipitated secretory proteins were dissolved in 9.8 M urea solution.

### (5) MALDI-IMS

Comparison of the distribution of metabolites on the surface of competition agar plates by MALDI-IMS revealed interesting differences in the ions secreted by the wild-type and mutants of P. taiwanensis. The regions of interest of the bacterial colonies were excised, and placed on glass slides. Slides with interesting target samples were covered with a thin layer of universal MALDI matrix (Sigma-Aldrich) deposited over the sample using a 50 μm sieve. The matrix-covered agar samples were dehydrated in an incubator at 37° C. overnight prior to IMS. The samples were analyzed by a Bruker Autoflex Speed MALDI-TOF/TOF MS and the data were collected. Samples were analyzed in positive reflectron ion mode, screened at 200 μm laser intervals with the acquisition mass range set at 100-2000 Da. The equipment was calibrated using a standard peptide calibration mixture (Peptide Calibration Standard 206195, Bruker, 1000-3200 Da) and matrix. The IMS data were analyzed using Fleximaging 3.0 software (Bruker). The intensity of molecules was presented as gradient colors.

### (6) Purification and Determination of Pyoverdine

The method of pyoverdine purification was modified from Yin et al. (Biosensors & bioelectronics 51, 90-96 (2014)). 50 ml of P. taiwanensis in 250 ml flask was incubated in iron-limited medium at 28° C. and 200 rpm for 24 h. The culture supernatant was collected by centrifugation at 4,600 g for 15 min at 4° C. and filtered through 0.22 μm sterile low protein binding polyvinylidene fluoride (PVDF) membrane filters (Millex-GV; Millipore). A chelating Cu-sepharose column was used to purify pyoverdine. Copper ions (Cu²⁺) used for recharging the sepharose from Ni-sepharose high performance (GE). 5 ml Ni-sepharose was loaded in 0.8×4 cm Poly-Prep chromatography column (Bio-Rad) and allowed buffer to flow through by gravimetric method. To remove residual Ni²⁺, Ni-sepharose column was washed with 5 column volumes buffer (0.02 M Na₂HPO₄, 0.5 M NaCl, and 0.05 M EDTA; pH 7.2). Then the column was washed to remove residual EDTA by at least 5 column volumes of distilled water and recharge sepharose with 0.5 ml of 1M CuSO₄. Consequently, the Cu-sepharose was washed with 5 column volumes binding buffer (0.02 M Na₂HPO₄, 1 M NaCl; pH 7.2).

The filtered culture supernatant was mixed with binding buffer in ratio of 1:1. 20 ml mixture was loaded in Cu-sepharose column to purified pyoverdine or other siderophores. The column was washed with 5 column volumes binding buffer again. Finally, siderophores were eluted by elution buffer (0.02 M Na₂HPO₄ and 1M NH₄Cl; pH 7.2) and dried by Freeze Dryer. The purified compound was checked by HPLC analysis with RP-Amide C16 column (4.6×250 mm, 5 μm; Sigma-Aldrich) and MALDI-TOF MS. The absorption maxima wavelength of fluorescent pyoverdine was evident within 407-412 nm. Here, the chromatography of HPLC was monitored over a range of 200-500 nm by UV absorption detector. The acetonitrile-water gradient of HPLC mobile phase was from 50% to 0% acetonitrile over 10 min at a flow rate of 1 ml/min Fractions were collected every minute and detected by MALDI-TOF. For identifying structural characterization, the peak of m/z 1044 was determined by ESI-Orbitrap (metabolomics core of Academia Sinica).

(7) Inhibitory Concentration (IC₅₀) and Lethal Dosage (LD₅₀) Assays

Purified pyoverdine dissolved in ½ TSB and sterilized by 0.22 filter. ½ TSB media containing pure pyoverdine from 5.5 to 0 mg/ml was placed in tubes containing 2 ml of ½ TSB. To study the effect of pyoverdine on growth of Xoo, absorbance at 600 nm and the number of viable cells (cfu/ml) were assayed after two nights incubation at 28° C. and 200 rpm. Assays were conducted in triplicate and consistent results were obtained.

### (8) CAS Plate Assay

Chrome azurol S (CAS) is a universal method that detects the mobilization of iron, which assays siderphores production. To prepare 100 ml CAS dye, 60.5 mg CAS powder (Sigma) was dissolved in 50 ml distilled water and mixed with 10 ml of 1 mM iron solution (anhydration FeCl₃, Alfa Aesar). Then, 40 ml of 72.9 mg HDTMA (Sigma) was added slowly to 60 ml CAS solution with FeCl₃ and autoclaved to sterilize. After CAS cool down can be hand held, one-tenth of CAS solution mixed with LP agar medium and immediately poured into plates.

CAS plates were used to demonstrate purified pyoverdine activity. Different concentration of purified pyoverdine was injected into the hole (5 mm) of CAS plates. Plates were incubated at 28° C. for 6 h or until yellow halo appearance.

### (9) Quantification of Subcellular Pyoverdine

Extracellular mature pyoverdine was quantified from cell free culture supernatant of P. taiwanensis after growing in iron-limited medium for 14 h. Culture supernatant was collected by centrifugation (6,000×g, 3 min) and filtered by a 0.22 μm pore size filter. To separate the periplasmic and cytosolic fractions, spheroplasts were obtained according to the method outlined in Imperi et al. (Proteomics 9:1901-1915, 2009). Cell pellets (3×10⁹ cells) were washed three times in PBS buffer (pH 7.4). The cell pellets were suspended in 1 mL of the spheroplasting buffer (10 mM Tris-HCl, pH 8.0, 200 mM MgCh, 0.5 mg/mL lysozyme), and incubated with gentle shaking for 30 min at room temperature. After incubation, the periplasmic fractions were collected by centrifugation (11,000×g, 15 min, 4° C.). The spheroplasts were washed three times in PBS buffer (pH 7.4). The pellets were suspended in 1 mL of sonicating buffer (10 mM Tris-HCl, pH 8.0, 100 mM NaCl) and lysed by sonication. After centrifugation (16,000×g, 5 min), cell debris was removed to obtain the cytoplasmic fractions. Mature fluorescent pyoverdine was determined using appropriate dilutions of dilution buffer (100 mM Tris-HCl) using a fluorescence Plate Reader (Victor 2, Perkin-Elmer) with excitation/emission wavelengths of 405/460 nm. Pyoverdine values were normalized against the cell optical density (OD600).

### Example 2: Treatment of Xoo-Infected Rice Leaves with P. taiwanensis

The japonica rice cultivar Tainung 67 (Oryza sativa L.) was used in pot experiments. We infected the leaves of 6-week-old plants with Xoo by the scissor-clip method. A P. taiwanensis culture supernatant or a P. taiwanensis culture was sprayed on the plants immediately after infection. After the first spray, the plants were sprayed three more times during a two-week period. Three weeks after infection, the treated leaves were significantly healthier than the untreated control leaves, which were dry and yellow.

### Example 3: Insecticidal Activity of Pseudomonas taiwanesis

We found that Pseudomonas taiwanensis is a broad-host-range entomopathogenic bacterium that exhibits insecticidal activity toward agricultural pests Plutella xylostella, Spodoptera exigua, Spodoptera litura, Trichoplusia ni and Drosophila melanogaster. Oral infection with different concentrations (OD=0.5 to 2) of wild-type P. taiwanensis resulted in insect mortality rates that were not significantly different (92.7%, 96.4% and 94.5%). The TccC protein, a component of the toxin complex (Tc), plays an essential role in the insecticidal activity of P. taiwanensis. The ΔtccC mutant strain of P. taiwanensis, which has a knockout mutation in the TccC gene, only induced 42.2% mortality in P. xylostella even at a high bacterial dose (OD=2.0). TccC protein was cleaved into two fragments, an N-terminal fragment containing an Rhs-like domain and a C-terminal fragment containing a Glt symporter domain and a TraT domain, which might contribute to antioxdative stress activity and defense against macrophagosis, respectively. Interestingly, the primary structure of the C-terminal region of TccC in P. taiwanensis is unique among pathogens. Membrane localization of the C-terminal fragment of TccC was proved by flow cytometry. Sonicated pellets of P. taiwanensis ΔtccC strain had lower toxicity against the Sf9 insect cell line and P. xylostella larvae than the wild type. We also found that infection of Sf9 and LD652Y-5d cell lines with P. taiwanensis induced apoptotic cell death. Further, natural oral infection by P. taiwanensis triggered expression of host programmed cell death-related genes JNK-2 and caspase-3.

Insecticidal Activity of TccC of P. taiwanensis Toward P. xylostella

In a previous study, the TccC gene from P. taiwanensis was overexpressed in E. coli and the recombinant TccC was able to increase the mortality in Drosophila larvae. See Liu et al., Journal of Agricultural and Food Chemistry 58: 12343-12349 (2010). In addition to Drosophila melanogaster, we found that P. taiwanensis has insecticidal activity against a number of Lepidopteran species, including several vegetable pests Plutella xylostella, Spodoptera exigua and Trichoplusia ni.

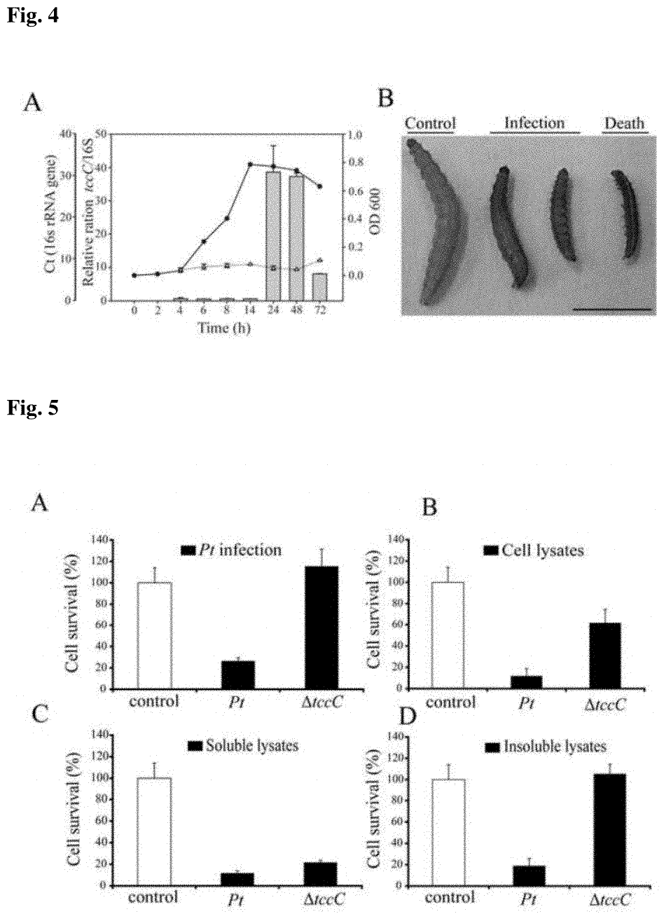

We investigated the in vivo insecticidal activities of the P. taiwanensis TccC against the Lepidopteran species P. xylostella. The expression level of TccC in P. taiwanensis was highest when bacterial cells reached the stationary phase (24 h) (FIG. 4A). Therefore, we collected P. taiwanensis cells at this stage and determined their toxicity. The P. taiwanensis cells were orally administered to the P. xylostella larvae. The larvae in the treatment group exhibited slower growth and were melanized, dehydrated, and rigid in comparison with those in the control group (FIG. 4B).

We compared the amino acid sequences of several TccC-like proteins from different pathogens, and found that all of them had an N-terminal conserved RhsA-like domain and a C-terminal hypervariable fragment. Interestingly, the TccC of P. taiwanensis has a unique sodium/glutamate symporter-like domain and a TraT-like domain in the C-terminal region. In order to evaluate the function of the TccC protein, we generated an isogenic tccC gene knockout mutant, designated ΔtccC, of P. taiwanensis. Table 1 shows the mortality rates of P. xylostella larvae orally administered with whole cells or different cell fractions of wild-type or ΔtccC P. taiwanensis. The mortality of P. xylostella larvae infected with P. taiwanensis ΔtccC strain (OD=2.0) was only 42.4% while those infected with wild-type P. taiwanensis was 94.5% (Table 1).

TABLE 1

| | % Treated | P value |
|---|---|---|
| Treatment^{a} | Mortality (n)^{b} | (two tailed)^{c} |
| Control | 1.8% (1/55) | P < 0.05 |
| Whole cells of P. taiwanensis^{e} | | |
| Wild-typestrain | | |
| OD = 0.5 | 92.7% (51/55) | P < 0.05 |
| OD = 1.0 | 96.4% (53/55) | P < 0.05 |
| OD = 2.0 | 94.5% (52/55) | P < 0.05 |
| ΔtccC mutant strain | | |
| OD = 2.0 | 42.4% (14/33) | P < 0.05 |
| Crude extract of P. taiwanensis^{f} | | |
| Wild-type strain | | |
| Cell lysates | 67.3% (33/49) | P < 0.05 |
| Insoluble lysates | 50.0% (24/48) | P < 0.05 |
| Soluble lysates | 31.3% (15/48) | P < 0.05 |
| Secretory proteins | 65.9% (29/44) | P < 0.05 |
| ΔtccC mutant strain | | |
| Cell lysates | 45.6% (21/46) | P < 0.05 |
| Insoluble lysates | 25.0% (11/44) | P < 0.05 |
| Soluble lysates | 32.7% (16/49) | P < 0.05 |
| Secretory proteins | 64.3% (27/42) | P < 0.05 |

^{a}P. taiwanensis wild-type, ΔtccC mutant strains, and their various proteins fractions were fed to three instar of healthy larvae.
^{b}Mortality is the percentage of larvae death. n is the sample size of the treated groups. The data were collected on day 5.
^{c}The two tail student t-test was used to elucidate statistical significance. Each treatment was repeated three times.
^{d}Similar to b, n is the sample size of the negative control PBS-treated group.
^{e}Ingestion dose: 50 μl OD = 0.5, 1, 2 cells/0.5 * 1 cm² vegetable block.
^{f}Ingestion dose: The crude extract contained 300 ng of protein.

We further prepared different cellular fractions of P. taiwanensis and tested their effects on P. xylostella larvae. More than 50% of P. xylostella larvae infected with cell lysates, insoluble lysates (cell membranes and cell wall pellets) and extracellular supernatants of wild-type P. taiwanensis died at the end of the 5-day feeding period (Table 1). Moreover, the mortalities of P. xylostella larvae infected with cell lysates and insoluble pellets of P. taiwanensis ΔtccC were lower than those infected with wild-type lysates (Table 1). These results indicate that the insecticidal activity of P. taiwanenesis might be attributable, at least in part, to the TccC.

Infection of Lepidopteran larvae with toxins, bacteria or viruses caused the appearance of apical protrusion and protrusion ruptures in the damaged enterocytes.

Therefore, we performed histological analyses to assess the effect of P. taiwanensis infection on the intestinal tracts of P. xylostella. The ultrastructure of the midgut of P. xylostella larva showed that oral infection with P. taiwanensis had a strong impact on gut cells. After infection with P. taiwanensis for 48 h, apical protrusion of enterocytes, abnormal microvilli and cell lysis were induced in the guts in P. xylostella indicating that P. taiwanensis infection caused serious injury to the midgut epithelial cells, which could not be repaired in the homeostatic process and finally caused the death of the host. Similarly, ultrastructure sections of P. xylostella larvae that ingested 100 ng toxin complex (Tc)/cm² food, showed columnar cells in the guts containing many vesicle-like structures. In contrast, ingestion of the ΔtccC mutant only showed abnormal microvilli without any apical protrusions or cell lysis.

Damage to the gut can induce stem cells to proliferate and differentiate to replace the damaged cells, producing a higher number of goblet cells with a larger shape than the control group. We observed that oral infection of P. xylostella with P. taiwanensis ΔtccC resulted in a greater number of goblet cells in the midgut system as compared with the non-infected or wild-type P. taiwanensis-infected P. xylostella indicating that only infection of ΔtccC, but not the wild-type, could induce the differentiation of damaged cells and the formation of many goblets in the midgut system. This suggests that the toxicity of P. taiwanensis ΔtccC was lower than that of the wild-type strain, and the midgut epithelial cells could be repaired in the process.

The colonization and invasion of midgut epithelial cells of P. xylostella by P. taiwanensis were further confirmed by bacterial quantification and histological examination.

After oral infection for 48 h, the bacterial counts of P. taiwanensis ΔtccC were lower than those of wild-type strain in the midgut of P. xylostella. In addition, the midgut epithelial cells were seriously disrupted by wild-type P. taiwanensis after oral infection for 48 h.

The insecticidal activity of the TccC was further confirmed by treatment of Sf9 insect cells with different P. taiwanensis cell fractions. See FIG. 5. The survival rates of Sf9 insect cells exposed to the intact cells (P. taiwanensis alive), cell lysate (total proteins), soluble lysate (cytosolic proteins) and insoluble lysate (cell wall and cell membrane) of wild-type P. taiwanensis were significantly lower than those exposed to PBS buffer. On the other hand, the survival rates of Sf9 insect cells exposed to the intact cells or cell wall pellets of P. taiwanensis ΔtccC were not significantly different from those exposed to PBS buffer, only those exposed to the cell lysates or soluble lysate of P. taiwanensisΔtccC were significantly decreased. Since P. taiwanensis ΔtccC did not express TccC, it was likely that some other virulence factors were present in the cell lysates of P. taiwanensis ΔtccC. Furthermore, active phagocytosis was found in Sf9 viable cells, a characteristic phenomenon during in vivo apoptosis but uncommon for in vitro cultures. Sf9 cells are phagocytic and contain unusually high numbers of phagosomes, particularly after glucose depletion. In the early infection stage (after incubation for 1 h), RFP-labeled P. taiwanensis was phagocytosed by Sf9 cells. After incubation for 3 h, lysis of Sf9 cells infected with P. taiwanensis was observed, as compared with no lysis in non-infected cells.

Induction of Apoptotic Cell Death by TccC of P. taiwanensis

To determine whether P. taiwanensis infection induces apoptosis in Lepidopteran Sf-9 and LD-5d cells, we used Annexin V-FITC to stain for apoptotic cells and DAPI staining to determine total cell numbers. Apoptosis was detected in Lepidopteran Sf-9 and LD-5d cells after 10 h of infection with P. taiwanensis and significantly higher mortality rates were observed than in the non-infection control. Furthermore, the JNK pathway of the gut epithelial cells of P. xylostella larvae was triggered by P. taiwanensis infection. In addition to the JNK pathway, we also examined the expression of the caspase genes, which can also induce apoptotic cell death. After 48-h oral infection with P. taiwanensis, the expression level of cleaved-caspase-3 was increased in the midgut cells. The expression levels of JNK-2 and cleaved-caspase-3 in P. xylostella larva infected with P. taiwanensis ΔtccC were lower than in the wild-type strain of P. taiwanensis, indicating that TccC might induce apoptosis and play an important role in cell death of the gut epithelial cells of P. xylostella larvae.

Effect of TccC on the Antioxidant Activity of P. taiwanensis

The digestive tracts of healthy insects are protected against bacterial disruption by an intact gut epithelial barrier and the host immune defense system. We analyzed the protease and antioxidative activities of P. taiwanensis strains to evaluate their resistance against the insect gut immune system. At the stationary phase of bacterial growth, P. taiwanensis secreted large amounts of proteases and showed high antioxidative activity. The antioxidative activity of P. taiwanensis ΔtccC was significantly lower than that of wild-type P. taiwanensis, indicating that the antioxidative activity of P. taiwanenesis might be directly or indirectly regulated by the TccC.

In order to confirm the involvement of the TccC in antioxidative activity, wild-type and ΔtccC P. taiwenansis were exposed to different concentrations of hydrogen peroxide and the bacterial counts were determined. The results showed that wild-type P. taiwenansis had a higher survival rate than ΔtccC, demonstrating that TccC also played a role in the protection of bacterial cells against ROS. ROS induces greater damage in the tccC mutant at high concentrations of H2O2 treatment. The P. taiwanensis TccC protein contains a sodium/glutamate symporter Glts—like domain in its C-terminal, which might function in glutamate transport. Since L-glutamate can be converted to glutathione, TccC might play a role in defense against ROS attack and maintain the intracellular redox potential in P. taiwanensis. We next determined whether P. taiwanensis possesses the ability to degrade hydrogen peroxide (H2O2). We found that 1 mM H2O2 was quickly degraded after incubation with wild-type P. taiwanensis for 2 min. In contrast, it took 15 min to completely decompose when incubated with tccC mutant. Together, our results suggested that wild-type P. taiwanensis has higher H2O2 detoxification activity, and can, therefore protect itself from ROS attack generated by the host immune response more effectiently than the tccC mutant.

### Antiphagocytic Activity of TccC

To evaluate the antiphagocytic activity of TccC, we performed a phagocytosis assay in which wild-type and ΔtccC P. taiwanensis cells were fluorescent-labeled with CFSE and then incubated with mouse macrophage cells. Macrophage cells incubated with fluorescent-labeled P. taiwanensis ΔtccC for 30 min showed a shift in the peak position toward higher fluorescence intensity, indicating that the amount of phagocytized ΔtccC was larger than that of phagocytized wild-type P. taiwanensis. To substantiate the findings of the scatter plot analysis, the percentage of phagocytized P. taiwanensis was calculated. The mouse macrophages engulfed fewer wild-type cells than the ΔtccC cells, suggesting that wild-type P. taiwanensis possessed antiphagocytic activity that might be partly attributable to TccC. We also analyzed the cytotoxicity of P. taiwanensis wild-type and ΔtccC toward mouse marcophages and found that the survival rate of mouse marcophages in the presence of the wild-type was not different from that in the presence of ΔtccC, suggesting that P. taiwanensis does not have a cytotoxic effect on mouse macrophages.

### Processing and Location of TccC In Vivo

Based on Pfam domain prediction, TccC is predicted to possess an RhsA domain (11-673), an Rhs repeat-associated core (600-680), sodium/glutamate symporter-like (726-825) and TraT complement resistance-like domain (736-781). In addition, three transmembrane regions (718-742, 744-758, 760-778) were predicted at the C-terminal region. Western blot analyses were performed to determine subcellular localization of TccC protein in P. taiwanensis. Three cellular fractions were prepared according to the method outlined in FIG. 6. Surprisingly, two protein bands were detected in the total cellular protein fraction, a ˜70 KD and a ˜40 KD bands, representing a processed form of TccC protein In the soluble protein fraction, only the ˜70 kD band was detected, whereas in the insoluble pellet fraction that contained cell wall and membrane proteins only the processed ˜40 kD band was detected. This suggests that TccC protein was processed when it was inserted into the membrane of P. taiwanensis cells.

We have observed that the recombinant TccC protein also was similarly processed in E. coli expression system. To further characterize the cleavage process, TccC with 6×His-tag was cloned into a broad host range vector pCPP30, and overexpressed in P. taiwanensis and E. coli (BL21). The His-tagged TccC proteins were purified using a nickel ion column. Western blot analysis showed that processed forms of TccC proteins with similar molecular weight were purified from both E. coli and P. taiwanenesis (Figure S8). This result suggests that the TccC has a similar cleavage site in E. coli and P. taiwanensis.

To test whether the TccC was indeed integrated into cell membrane, the TccC was labeled with FITC to trace the outer membrane fraction by staining with TccC-FITC antibody. Flow cytometry analysis showed that the fluorescence signal of TccC on the cell surface of P. taiwanensis had significantly higher density than the non-stained control. In contrast, no significant fluorescence density was detected in the tccC mutant.

### Materials and Methods

### (1) Bacterial Strains, Culture Condition, and Antibiotics

P. taiwanensis BCRC 17751 was used as the entomopathogenic species. Escherichia coli DH5a was used in all construction experiments. E. coli S17-1 was used for biparental mating with P. taiwanensis, and E. coli BL21 was used to express recombinant protein. P. taiwanensis and E. coli were grown in Luria-Bertani (LB) broth or on an agar plate. P. taiwanensis cultures were grown at 30° C. and E. coli cultures were grown at 37° C. Antibiotics were applied at the following concentrations: rifampicin (34 □g/ml), ampicillin (100 □g/ml), and spectinomycin (100 □g/ml) for P. taiwanensis wild-type cultured media; and kanamycin (30 □g/ml), tetracycline (20 □g/ml) for P. taiwanensis mutant strain and overexpression strain, respectively; kanamycin (50 □g/ml), ampicillin (100 □g/ml), and tetracycline (20 □g/ml) for E. coli strain.

### (2) Cell Culture

Both the Lepidoptera insect Spodoptera frugiperda Sf9 cell line and Lymantria dispar IPLB LD-652Y-5d cell line were provided by Dr. C. H. Wang (Department of Entomology, National Taiwan University). The gypsy moth (Lymantria dispar) cell line, IPLB LD-652Y-5d was subcloned from IPLB LD-652Y [47]. They were grown in Sf-900 II SFM (Gibco) medium supplemented with 10% fetal bovine serum (FBS) and 1% penicillin/streptomycin/glutamine (PSG) (Invitrogen) at 27° C.

(3) Construction of the P. taiwanensis ΔtccC Knockout Mutant

An tccC (GenBank database accession number, HQ260745) knockout mutant of P. taiwanensis, designated ΔtccC was constructed by double recombination of the suicide vector pEX100T containing the tccC fragment with a kanamycin resistance cassette inserted. A tccC-kan-tccC fragment was generated by inserting a 1345-bp kanamycin resistance cassette into an 852-bp fragment that contains the coding sequence of tccC. The tccC-kan-tccC fragment was cloned into pEX100T suicide vector, and then transformed into E. coli S17-1 for conjugation with wild-type P. taiwanensis. The double recombination tccC mutant strain was selected on LB plates containing 5% sucrose, 30 □g/ml kanamycin, 34 □g/ml rifampicin, and 100 □g/ml spectinomycin. The resulting ΔtccC mutant was confirmed by PCR and sequencing.

### (3) Bioassay of Infection Experiments and Effective Protein Fractions

Bioassays of bacteria infection of larvae were performed by natural oral infection. P. taiwanensis was grown for 24 hours to the stationary phase and collected. Subsequently, the cell pellet was washed three times in 5 ml PBS (pH 7.4) and resuspended in PBS, adjusted to different concentrations (OD). Different concentrations of bacteria (50 μl) were applied to surface of 0.5×1 cm² vegetable pieces, which were used for feeding larvae of vegetable moth Plutella xylostella and incubated at 25° C. Each infected larva was observed at day 5 after oral infection and the mortality rate was calculated. Healthy third-instar P. xylostella larvae were provided by the Taiwan Agricultural Chemicals and Toxic Substances Research Institute. To determine the protein fractions that cause mortality against P. xylostella, P. taiwanensis was cultured for 24 hours. The cell culture was harvested by centrifugation (15 min at 4,600 g, 4° C.), and supernatants and cell pellets were collected separately. For culture supernatants, the secreted proteins were filtered through a 0.22 □m PVDF filter (Millipore) and concentrated using a Vivaspin 20 concentrator (10 kDa MWCO, GE Healthcare). The harvested cell pellets were washed with PBS two times and resuspended in PBS with protease inhibitor and lysed with sonication (cell lysates). The cell lystaes were separated into insoluble lysates and soluble lysates by centrifugation (30 min at 26,000 g, 4° C.), and the soluble lysates were filtered by a 0.22 □m PVDF filter. The insoluble lysates were washed with PBS two times and resuspended in PBS. For toxicity analysis of protein fractions from P. taiwanensis, 300 ng of proteins dissolved in 10 □l PBS were used for insect larvae treatment. Protein extracts were quantified by Pierce 660 nm protein assay method (Pierce).

### (4) Cell Survival Assay

To investigate the effect of P. taiwanensis on insect cells, proliferation of Spodoptera frugiperda Sf9 cells was determined by a colorimetric XTT assay. For cytotoxicity assay, Sf9 cells were seeded at 5,000 per well in 96-well culture plates supplemented with 10 μg/ml of the various fraction proteins of P. taiwanensis or a multiplicity of infection (MOI) of 1000 Pt/cell was added in antibiotic-free medium. After 72-h treatment, cell proliferation was quantified by Cell Proliferation Assay Kit (XTT) (Biological Industries).

### (5) Apoptotic Assay

Cell early stage apoptosis was detected by Annexin V-FITC assay. The percentages of apoptosis of human or insect cells were determined by counting visible annexin V-positive cells under the fluorescence microscope. Cells (5,000 cells/well) were incubated with protein fractions of P. taiwanensis at 10 μg/ml or with P. taiwanensis (MOI=1000) for 72 h on the well in 24-well plates. After treatment for 72 h, the cells were washed twice in PBS and detected using the ApoAlert Annexin V-FITC Kit (BD) according to the manufacturer's instructions. The DNA in the nuclei was stained with 4′,6-diamidino-2-phenylindole dilactate (DAPI) for 5 min Finally, the stained cells were washed twice in PBS, fixed with 4% paraformaldehyde for 10 minutes, and then observed under a fluorescence microscope (Zeiss Axiovert 100M, Carl Zeiss, Germany). Annexin V positive cells were counted and identified as P. taiwanensis-induced early stage apoptotic cells.

### (6) Sectioning and HE, Gram, Immunohistochemistry Staining

After bacteria oral infection for 48 h, third instar larvae were fixed in 10% buffered formalin (pH 7.0) for at least 48 h. After fixation, larvae were sent to the Laboratory of Pathological Section of National Taiwan University for sectioning. The tissue sections were analyzed by hematoxylin-eosin, Gram's, or immunohistochemistry staining. Immunohistochemical (IHC) staining was performed using anti-JNK-2 [N1C3] (GTX105523, Genetex; 80% [276/398] sequence identity to c-Jun NH2-terminal kinase of Bombyx mori, NP_001103396) and anti caspase-3 p17 (GTX123678, Genetex; 36% [ 46/129] sequence identity to caspase 3 of Bombyx mori, AAW79564) antibodies, followed by diaminobenzidine (DAB) for color development and counterstained with hematoxylin from the Laboratory Animal Center of National Taiwan University Hospital.

### (7) Purification of TccC

Full-length TccC-His₆ fusion fragment was cloned into the broad host range Pcpp30 vector and transformed into E. coli (BL21) and P. taiwanensis. Overexpressed TccC-His₆ fusion protein was purified by His SpinTrap columns (GE Healthcare) after P. taiwanensis and E. coli growth into stationary phase (24 h), and the results were displayed by western blotting using the anti-TccC antibody.

### (8) Analysis of TccC Location

For SDS PAGE, 20 □g proteins of different cellular fractions from P. taiwanensis were dissolved in loading buffer with SDS and then applied to gel electrophoresis. After electrophoresis, the proteins were transferred to nitrocellulose membranes under 40 mA for 12 h. TccC was detected with specific anti-TccC antibody, using rabbit polyclonal antibodies raised against P. taiwanensis TccC full-length recombinant protein purified from E. coli BL21 expression. After first antibody binding, the color was developed with horseradish peroxidase-coupled anti-rabbit secondary antibody binding and chemoluminescent detection reagent (Pierce).

Flow cytometry was used to determine membrane localization of TccC. Wild-type and ΔTccC mutant strains of P. taiwanensis were grown overnight and collected at stationary phase (24 h). The cultures were adjusted 10⁹ CFU/ml, and then 100 l adjusted-bacteria was centrifuged to collect pellets. The bacteria pellets were washed three times with PBS at 4° C. and resuspended in 200 □l PBS with 1% BSA. The polyclonal anti-TccC antibody (1/100 dilution) was added to the bacteria suspension on ice for 1 h. The bacteria was washed three times with PBS again and stained with goat FITC-conjugated anti-rabbit IgG secondary antibody (1/100 dilution) (Jackson Immunoresearch) on ice for 1 h. After staining, the bacteria were washed three times and resuspended in 1 ml PBS and analyzed by flow cytometry. Flow cytometry was performed by MoFlo XDP Cell Sorter (Beckman Coulter) using Summit 5.2 software (Beckman Coulter).

### (9) Phagocytosis Assay

P. taiwanensis cells were collected in the early stationary phase and washed twice with PBS, and resuspended in PBS to OD=1 (4×109 cells). One milliliter of resuspended cells was added to CFSE (final concentration of 5 □M) and incubated at 30° C. in the dark for 30 min. The cells were washed three times with PBS and observed under fluorescent microscope. For phagocytosis assays, CSFE labeled P. taiwanensis cells were added to macrophage cells (MOI=1000) for 30 min at 37° C. in the dark, and then washed three times with PBS. Quantification and observation of phagocytosis was measured by flow cytometry and fluorescent microscope respectively. Flow cytometry was performed by Cytomics FC500 (Beckman Coulter) using CXP software (Beckman Coulter). Ten thousand cells were collected for analyses. Non-infected macrophage cells were used as a negative control.

(10) Quantitative H2O2 Assay and Proliferation Assay of P. taiwanensis

P. taiwanensis cells grown to stationary phase (24 h) were collected, washed three times in PBS, and resuspended in PBS to 10⁹ cells per ml and subsequently incubated with 1M H2O2. The concentration of H2O2 remaining was detected at different time points after treatments using a PeroX-Oquant Quantitative Peroxide Assay Kits (Pierce). Visualization of the proliferation effect of hydroxyl radicals in P. taiwanensis was performed as described previously. P. taiwanensis was grown in LB broth for 24 h and then incubated with different concentrations of H₂O₂ for 3 h. Proliferation was determined by counting the colony-forming units.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the described embodiments, and without departing from the spirit and scope thereof, can make various changes and modifications of the embodiments to adapt it to various usages and conditions. Thus, other embodiments are also within the claims.

What is claimed is:

1. A method of producing a composition for inhibiting growth of a microorganism, the method comprising culturing a Pseudomonas taiwanensis strain in a nutrient-limited medium to obtain a culture broth and collecting the culture broth, thereby producing the composition.

2. The method of claim 1, wherein the medium is an iron-limited medium.

3. The method of claim 2, wherein the medium is M9 minimal medium supplemented with casamino acids, MgSO4, and glycerol.

4. The method of claim 3, further comprising removing cells from the culture broth to obtain a cell-free supernatant and collecting the cell-free supernatant.

5. The method of claim 1, wherein the Pseudomonas taiwanensis strain has deposit number DSM 21245.

6. The method of claim 1, wherein the Pseudomonas taiwanensis strain has a loss-of-function rpoS mutation.

7. The method of claim 1, wherein the microorganism is a phytophathogenic bacterium, a phytophathogenic fungus, or a multidrug resistant bacterium.

8. The method of claim 7, wherein the microorganism is selected from the group consisting of Xanthomonas oryzae pv. Oryzae, Colletotrichum gloeosporioides, Phytophthora capsici, Pyricularia oryzae, Rhizoctonia solani, Fusarium oxysporum f sp cattleyae, Staphylococcus epidermidis, Staphylococcus aureus, or Candida albican.

9. A composition for inhibiting growth of a microorganism, wherein the composition is produced by the method of claim 1.

10. The composition of claim 9, further comprising one or more other anti-bacterial, anti-fungal, or insecticidal agents.

11. A method of inhibiting growth of a microorganism, the method comprising contacting the microorganism with the composition of claim 9.

12. The method of claim 11, wherein the microorganism is a phytophathogenic bacterium, a phytophathogenic fungus, or a multidrug resistant bacterium.

13. The method of claim 12, wherein the microorganism is selected from the group consisting of Xanthomonas oryzae pv. Oryzae, Colletotrichum gloeosporioides, Phytophthora capsici, Pyricularia oryzae, Rhizoctonia solani, Fusarium oxysporum f sp cattleyae, Staphylococcus epidermidis, Staphylococcus aureus, or Candida albican.

14. A method of treating or reducing the risk of rice bacterial blight, the method comprising applying to a rice plant in need thereof the composition of claim 9.

15. A method of inhibiting growth of a microorganism, the method comprising: contacting the microorganism with an isolated pyoverdine having the structure of Q-DSer-Lys-OHHis-aDThr-Ser-cOHOrn, wherein Q is a chromophore and the microorganism is a phytophathogenic bacterium, a phytophathogenic fungus, or a multidrug resistant bacterium.

16. The method of claim 15, wherein the microorganism is selected from the group consisting of Xanthomonas oryzae pv. Oryzae, Colletotrichum gloeosporioides, Phytophthora capsici, Pyricularia oryzae, Rhizoctonia solani, Fusarium oxysporum f sp cattleyae, Staphylococcus epidermidis, Staphylococcus aureus, or Candida albican.

17. The method of claim 16, wherein the microorganism is Xanthomonas oryzae pv. Oryzae.

18. A method of inhibiting growth of an insect, the method comprising contacting the insect with a composition containing a Pseudomonas taiwanensis strain, a Pseudomonas taiwanensis cell lysate, or a Pseudomonas taiwanensis TccC polypeptide, wherein the insect is a Lepidopteran species.

19. The method of claim 18, wherein the insect is Plutella xylostella, Spodoptera exigua, or Trichoplusia ni.

20. The method of claim 18, wherein the cell lysate is a whole cell lysate or a soluble lysate.

21. The method of claim 20, wherein the Pseudomonas taiwanensis strain is cultured in a nutrient-rich medium and the cell lysate is obtained from a Pseudomonas taiwanensis strain cultured in a nutrient-rich medium.


SEQUENCE LISTING (4 sequences)

SEQ ID NO: 1 - DNA, 3522 bp, Pseudomonas taiwanensis
  gene (1)..(3522): TccC
tactcatctgagtacgacagggatgccgccatgcctggcggcttttccgatacgtcaaacagcgctttccgactagcagtcagccatacagccaaatcaagctgattcttcactcccctctgggggtggcgaaaaatcaacatgatcaaggtaactgcaagttgggacacatagacttttcacttcataacggaacgcctacggtcaccgtccgagacaaccgaggattaggcatccgcgatatcgcttatcatcgccatcccgatacacccgaacaactcgacgaacgcatcacccgccaccggttcaacgcccttgcgcagcttgagcaaagcatcgatcctcgcctccatgaacgccaagccgtggacgcgacgacccaacccaattacaaatttcataattcgctgacgggcgatgtcctgcgtagcgacagtgccgacgcgggcgtcacgctctcgctcaacgatgttcacggccgcccgtgcctgagcattggcgccacaggcgcgctccatcgctggcactatgaaaccccaccgcttgcagggcgattgctacacgtgagtgagcatatcgccgaagcaaatccgcgcatcacagaacgcttggtctggggcgacaacacccagactgcgaaggatcagaatcttgcaggccgatgcgtgcgccactatgacacggcaggttgttggcagatggacagcgccggcttgtccgcaagcgtactttccgccacccaaaagttgctggcggaaggcaccgaagccgattggcagggagaggacgcggcagtctgggacaagctactagcaccggatgcgttcacgacctcacatcgtatcgacgcgacgggagcttccatcgagcaacgcgatgcgctcggccatacccaatgccaggcctatgacatagcgggcatgctgcgtagcactcggctgatcatgaaaggtggaacgacgcgggttatcttgaaggctgtggaatactccgcgttcggacaaaagctgcgggaagaacagggcaacggcgtcattaccacctacacctacgaacagcggactcagcgcctcctaggcagcaagatcgaacgacgtgccgggcgcagcgaggcgaaagtcctgcaagacatacggtacgaatatgacccagtcggtaatatcctgagtgtgcacaatgatgcggaggcgacgcggttctggcgtaatcagaagatcgtaccggtcaatcgctatgcatatgacagcctctatcaactgatctcagccagcggccgtgaaatggccgatatgccccgccaaggccctaagcctccctcccccaccattccactcccgaccaacgacggggcctacaccaactacacacgtcgttaccaatacgatcgcgctggcaacctgacgcgtatctcacacagcgcacccgcctccaacaacagctataccctggacatgacagtgtccaaccgcagcaatcgggcggtattgcatactctcgccgacgatcctgccaaggtcgatgccctcttcgatgcagcgggcaatcagttacaactgcaacctggccaatcccttcattggacaccgcgcgggcagctcggcaagttcgtgtcacaggcaggtgatgacagcgctgttgaccaggaaagctatcgctacggtgcagacggccaacggatcgccaaatacaactcccaacaggcaggcgcccaaacgggatacgtactttatttgccagggttggaggtgcgcgcccgtttcagggacgatgcgataaaagaactgcttcacgtgatcaccatcggcgaagccggtaatgctcaagtgcgattactgcactgggaaaccggcacaccgccaagtgtcagcaatgactcgctgcgctacggctattccaatctcatagacagcgtcgggctcgaactcgatagcgacggtcaaatcatcagctatgaagagtactacccctacggcggctctgcggtatgggccgctcgcagccagacagaagccgattacaagaccgtgcgttattcagggaaagaacgcgatggcacggggctctattattacggtcaccggtattaccaaccctgggtcgggcgctggctcagtgcagaccctgccggtacggtcgatggactcaatctctaccgaatggtacgaaacaaccccattgccttaaaagacaacaacggattgaatgccgaagggtattaccatgagttccaagcgctgaagagcgcacccagtatgatccgtaataccaggcttcaaattcaagattatatgcgaagccaaaccgaaagccggattatttacgtgttgatgtcggtcgttttggaagcgctcgctacgaccattggcatggccggcggcctcctgggcggtgcggcgggaggggctataggaggcgctgtaggaggggttatcgccaacgttccaggagccgctgtaggcgcaacctggggggctagcgtaggagggctcgtcgggaaaaccgttgtaaagaaagcggcagagaaaatactcccgcaggctgagttgacgccagacctcgacatgacaaaaaaaataaacgaaacggccgaaggcggccttaggcataaaatcaaacatttcctagaaaaagaaataaccatggaaaagctccgtggaaaaataaccgatgatcaaatgaccaacgatgcaaacaaagtggcgacaggcgtgggtttaccacaataccctcttacccttcccgtatcaaaagcgataaaagtcgccacagaagtggaaaaatcaataaccgttaccacaaaacatgcagtagccggggcaatacctgctcaagtagagattgcaaaaggtgcccttcatgccatttactcaaagatagacgcgcaattcggtaagctcagcagcatgcgcagccgtaaaagcctgttgaggcctttcatacccgatggcccacgagagctttccattacattgaataatgacccgttcaaccctgatgcatgggtgggaagatcggaggtcgagaagccttaccaggcagccttggccgaactggataaacttaacgaactgtacgttaagtacgaaaaaaaatttcgtacttaagcgatctcaacaaccggccccgccggtttgctgcatgcaagaccggcggtaccccaatgcctgaactcaccccgcctcagcccgaatccgtatcgcatcatgacgccaatattccaggtcacagtcgatcagatgcccatactggtcgctgttgacccgggtgacatgcaaccccggactacccgccgagaccttgagggctgcggcagccggagccggcaacgcggtcggcaggatctcgaagcatacccggccgtaagcgatcccataggctttggcatagatctcggtcagcgactgaccaagatccaactccaggatcccaggaaaatacctagggttcaggtaatgctcggcatacagcaccgcgcgcccgtcgatacgccgcaagcggcagatctgcaccacgctggacaac

SEQ ID NO: 2 - protein, 980 aa, Pseudomonas taiwanensis
  PEPTIDE (1)..(980): TccC
MGHIDFSLHNGTPTVTVRDNRGLGIRDIAYHRHPDTPEQLDERITRHRFNALAQLEQSIDPRLHERQAVDATTQPNYKFHNSLTGDVLRSDSADAGVTLSLNDVHGRPCLSIGATGALHRWHYETPPLAGRLLHVSEHIAEANPRITERLVWGDNTQTAKDQNLAGRCVRHYDTAGCWQMDSAGLSASVLSATQKLLAEGTEADWQGEDAAVWDKLLAPDAFTTSHRIDATGASIEQRDALGHTQCQAYDIAGMLRSTRLIMKGGTTRVILKAVEYSAFGQKLREEQGNGVITTYTYEQRTQRLLGSKIERRAGRSEAKVLQDIRYEYDPVGNILSVHNDAEATRFWRNQKIVPVNRYAYDSLYQLISASGREMADMPRQGPKPPSPTIPLPTNDGAYTNYTRRYQYDRAGNLTRISHSAPASNNSYTLDMTVSNRSNRAVLHTLADDPAKVDALFDAAGNQLQLQPGQSLHWTPRGQLGKFVSQAGDDSAVDQESYRYGADGQRIAKYNSQQAGAQTGYVLYLPGLEVRARFRDDAIKELLHVITIGEAGNAQVRLLHWETGTPPSVSNDSLRYGYSNLIDSVGLELDSDGQIISYEEYYPYGGSAVWAARSQTEADYKTVRYSGKERDGTGLYYYGHRYYQPWVGRWLSADPAGTVDGLNLYRMVRNNPIALKDNNGLNAEGYYHEFQALKSAPSMIRNTRLQIQDYMRSQTESRIIYVLMSVVLEALATTIGMAGGLLGGAAGGAIGGAVGGVIANVPGAAVGATWGASVGGLVGKTVVKKAAEKILPQAELTPDLDMTKKINETAEGGLRHKIKHFLEKEITMEKLRGKITDDQMTNDANKVATGVGLPQYPLTLPVSKAIKVATEVEKSITVTTKHAVAGAIPAQVEIAKGALHAIYSKIDAQFGKLSSMRSRKSLLRPFIPDGPRELSITLNNDPFNPDAWVGRSEVEKPYQAALAELDKLNELYVKYEKKFRT

SEQ ID NO: 3 - DNA, 1008 bp, Pseudomonas taiwanensis
  gene (1)..(1008): rpoS
atggctctcagcaaagaagtgccggagtttgacatcgacgatgacctcctgttgatggagacgggcatcgttttggaaacggatgtggtgtcagacgaacctgctgtaccttcggttcggaccaaggccaaacaaggctcatcgctcaaacagcacaagtacatcgattacagccgggcgctcgacgccacccagctgtatctcaacgaaatcggcttttctccgctgctctcccccgaagaggaagtgcattacgcacgcctgtcgcaaaaaggcgatccggctggccgtaagcgcatgatcgagagcaacctgcgcctggtggtcaagattgcgcgccgctacgtcaatcgtggcctgtcgctactcgacctgatcgaagagggcaacctcggtctgatccgcgcggtagaaaagttcgatccggagcgcggtttccgtttctcgacctatgcgacctggtggattcgccagaccatcgaacgggcgatcatgaaccagacccgcaccatccgcctgccgatccacgtggtcaaggagctcaacgtctacctgcgtgccgcgcgggagctgacccagaagctcgaccacgagccttccccggaagaaatcgccgggcttttggaaaaacccgtggccgaggtcaagcgcatgcttgggctcaacgagcgtgtctcttcggtggacgtttctctcggcccggactccgacaagaccctgctcgacacgctgacggacgatcgcccgaccgacccgtgcgagctgctgcaggacgacgacctctcccagagcatcgaccaatggctgggtgagttgaccgacaagcagcgtgaggtggtggtgcgtcggttcggcttgcggggccacgaaagcagcacccttgaggatgtaggcctggaaatcggcctgacccgagagcgcgtgcggcagatccaggtcgaggggctcaagcgtctacgtgaaatccttgaaaagaacggcctctcgagtgagtcgctgttccagtaa

SEQ ID NO: 4 - protein, 335 aa, Pseudomonas taiwanensis
  PEPTIDE (1)..(335): rpoS
MALSKEVPEFDIDDDLLLMETGIVLETDVVSDEPAVPSVRTKAKQGSSLKQHKYIDYSRALDATQLYLNEIGFSPLLSPEEEVHYARLSQKGDPAGRKRMIESNLRLVVKIARRYVNRGLSLLDLIEEGNLGLIRAVEKFDPERGFRFSTYATWWIRQTIERAIMNQTRTIRLPIHVVKELNVYLRAARELTQKLDHEPSPEEIAGLLEKPVAEVKRMLGLNERVSSVDVSLGPDSDKTLLDTLTDDRPTDPCELLQDDDLSQSIDQWLGELTDKQREVVVRRFGLRGHESSTLEDVGLEIGLTRERVRQIQVEGLKRLREILEKNGLSSESLFQ